(19) United States
(12) Patent Application Publication
(10) Pub. No.: US 2022/0401308 A1
(43) Pub. Date: Dec. 22, 2022

(54) METHODS AND DEVICES FOR DISPENSING MEDICATION

(71) Applicant: Lenore Everson, Wayzata, MN (US)

(72) Inventor: Lenore Everson, Wayzata, MN (US)

(21) Appl. No.: 17/844,325

(22) Filed: Jun. 20, 2022

Related U.S. Application Data
(60) Provisional application No. 63/212,387, filed on Jun. 18, 2021.

(51) Int. Cl. A61J 7/04; G16H 20/13; G01N 33/483

(52) U.S. Cl. CPC A61J 7/0418; G16H 20/13; G01N 33/483; A61J 2200/30; A61J 2200/70

(57) ABSTRACT

Medication dispensing devices and methods of using such devices for identifying patients and automatically dispensing medications to the patients in a secure manner. Such devices may include a medication storage unit, a medication dispensing unit, a user interface, a user identification element, and a processor. The device may further include an enclosure defining a patient area at which a patient may interact with the machine portion of the medication dispensing device. Such medication dispensing devices may be particularly useful for medications which require daily delivery of a medication in a controlled manner, such as methadone and suboxone.

### BACKGROUND OF THE INVENTION

It can be difficult for some patients to obtain necessary medications in a safe and timely manner. For example, for medications which have the potential to be abused, only small numbers of doses may be dispensed to a patient at one time. In the case of methadone or suboxone, many patients can only receive a single dose at a time, requiring them to visit a dispensing location every day. However, there are numerous regulations around the dispensing of methadone and suboxone. In addition, neighborhoods often oppose the establishment of “methadone clinics” in their area out of fear of associated crime. As a result, there are few locations which are able to provide the daily dose to patients, and some patients must travel long distances each day to receive their medication or wait in long lines with other patients. For some patients who must travel great distances to a dispensing location, such transportation may be provided by the government at significant expense.

Methadone and suboxone are examples of medications which, in many cases, must be provided to patients on a daily basis and with strict regulatory controls. Methadone and suboxone are used to treat patients with opiate addiction such as heroin addiction, to reduce symptoms of withdrawal as well as for maintenance to prevent relapse. However, because methadone and suboxone are themselves narcotics, there is a risk that patients will take too much of the prescribed medication, or will take it in combination with alcohol or other drugs, resulting in an overdose, or give or sell the medication to others. As a result, particularly in the early stages of treatment, regulations require that patients only receive their daily dose one day at a time. In addition, there is an illegal market for methadone and suboxone, for use as recreational drugs. Therefore, there is a significant risk of theft. There is also a risk of diversion, by which a patient does not consume the medication but rather saves it to sell or give to others, either for recreational use or to help with other people's symptoms of withdrawal. The regulations therefore further require that the medication be consumed immediately, under observation, to prevent diversion.

As a result, it may be difficult to access medications such as methadone or suboxone, particularly for rural patients and others who live far from a dispensing location. The stigma associated with such clinics, and fear of criminal activity, may also suppress use of the medication by those who could benefit from it.

### BRIEF DESCRIPTION OF THE DRAWINGS

The following drawings are illustrative of embodiments and do not limit the scope of the invention. The drawings are not necessarily to scale and are intended for use in conjunction with the following detailed description. Embodiments of the invention will be described with reference to the drawings, in which like numerals may represent like elements.

FIG. 1 is a schematic diagram of a medication dispensing device.

### SUMMARY

Various embodiments include medication dispensing devices including a medication storage unit, a medication dispensing unit, a user interface, a user identification element, and a processor, wherein the medication dispensing device is configured to identify a patient and dispense one or more doses of a medication. The medication dispensing device may be configured only a single dose of the medication and to confirm that the patient consumed the single dose of medication immediately. In some embodiments, the medication dispensing device may include a medication spoliation unit configured to ruin the stored medication when an attempted theft is detected. In some embodiments, the user identification element may be a camera, fingerprint scanner, a retina scanner, a bar code scanner, or a QR code scanner.

In some embodiments, the medication dispensing device may include a camera. In such embodiments, the medication dispensing device may include a wired or wireless data output, and the device may be configured to provide images or video of a patient interacting with the device or consuming the medication to a remote observer.

In some embodiments, the medication dispensing device may include a specimen collection unit. The specimen collection unit may be a breath analyzer, for example.

In various embodiments, the medication dispensing device include a machine portion and an enclosure defining a patient portion. The machine portion includes a medication storage unit, a medication dispensing unit, a user interface, a user identification element, and a processor. The medication dispensing device is configured to identify a patient and dispense one or more doses of a medication.

In some embodiments, the enclosure completely surrounds the patient area. The enclosure may include a door secured by a lock providing controlled entry to the patient area. In some such embodiments, the lock may be a user identification element such as a finger print scanner, a retinal scanner, a key pad, a bar code scanner or a QR code scanner.

In some embodiments, the medication dispensing device may also include a medication spoliation unit configured to ruin the stored medication when an attempted theft is detected.

Various embodiments include methods of dispensing medication using the medication dispensing devices such as those described above. The method includes storing medication in the mediation dispensing device, receiving data from a patient, confirming the patient identity using the received data, and, after confirming the patient identity, automatically dispensing medication to the patient.

In some embodiments, the method further includes receiving a specimen from a patient, such as a breath sample or a urine sample. The method may further include analyzing the sample to obtain a result. The step of automatically dispensing the medication to the patient may only occur if the result of the sample analysis falls within a predefined parameter.

In some embodiments, the method includes receiving a video of the patient consuming the dispensed medication using a video camera in the device. The method may further include transmitting the video to a remote observer.

### DETAILED SUMMARY OF THE INVENTION

The following detailed description is exemplary in nature and is not intended to limit the scope, applicability, or configuration of the invention. Rather, the following description provides practical illustrations for implementing various exemplary embodiments. Utilizing the teachings provided herein, those skilled in the art may recognize that many of the examples have suitable alternatives.

Many medications, particularly medications which are subject to abuse or diversion such as medications in the opioid family, require secure storage and patient delivery. In some cases, this results in elaborate and expensive systems which can be difficult for patients to access. Various embodiments described herein include medication delivery devices which can securely dispense medications directly to patients at locations which are safe and convenient and in compliance with federal, state and local regulations.

Various embodiments include standalone medications dispensing devices which automatically identify patients and dispense medications as prescribed. These medication dispensing devices may be in the form of machine kiosks which may be installed in pharmacies. The devices may optionally include an enclosure such as walls and optionally a ceiling to provide enhanced patient privacy. In such cases, the patient may enter the enclosed medication dispensing device into a patient area where the patient may interact with the dispensing portion of the medication dispensing device. The devices may be any size adequate to include all of the functional components and comfortably facilitate a patient interaction. Enclosed embodiments may be sized to provide a patient area with an adequate size to comfortably fit an adult person inside the patient area, such as providing a space of at least about 6 feet in height, such as about 6 to about 8 feet or to about 9 feet in height, and at least about 2 feet in horizontal dimensions (in width and depth), such as about 2 feet to about 5 feet.

The medication dispensing devices may be used with various types of medications. The medication dispensing devices may be particularly useful for dispensing medications which must be delivered frequently, such as in daily doses. Example of such medications include methadone and suboxone, which are frequently dispensed to patients one day at a time to control their use and prevent diversion.

The medication dispensing devices may include patient identification units to identify the patient at the time of medication delivery and to ensure that only the correct person is receiving the medication. Prior to dispensing the medication, the patient identification unit may identify the patient using one or more methods such as a fingerprint scan, a retinal scan, a patient identification number, facial recognition technology, voice recognition technology, or other unique identification methods. In some embodiments, the medication dispensing device may include one or more cameras, fingerprint scanners, retinal scanners, microphones, or other components for patient identification. In other embodiments, one or more components used for patient identification may be provided by a mobile device such as a smart phone. For example, the camera, fingerprint scanner, or microphone of an individual patient's smart phone may be used to identify the patient using a smart phone app and/or through communication with a remote individual. A unique identifier may then be provided to the user, through the smart phone such as through the app, which the patient may input into the medication dispensing device for identity verification.

In addition to verifying the patient's identity, the medication dispensing device may collect specimens from the patient and/or perform analysis of the patient specimens to ensure that the patient is qualified or meets the requirements for receiving the medication prior to dispensing the medication. For example, the device may include a breath analysis unit, like a breathalyzer, into which a patient may breathe and which may detect the presence of substances such as alcohol. In some embodiments, the device may include a receiving unit for receiving patient specimens such as urine, which may be immediately tested by the medication dispensing device or may be stored by the medication dispensing device for later testing outside the device. For example, the urine may be tested for the presence of illegal substances such as narcotics, cannabis, or other substances. In some cases, the medication dispensing device may refuse to dispense medication if a particular substance is detected. For example, if the breath analysis unit detects the presence of alcohol, or if it detects the presence of alcohol above a particular limit which may be zero or may be greater than zero, it may not dispense medication to the patient at that time. The patient may have to return later that day, or the patient may have to wait until the next day, to return to the medication dispensing device and repeat the breath analysis and, if the requirements are met, to receive the medication. The medication dispensing device may also send a report of the results of the specimen analysis to health care providers such as physicians, therapists or others involved in the patient's care.

In addition, because some medications are subject to theft, the medication dispensing devices may include mechanisms to deter or prevent theft and/or spoil any stollen medication. For example, the medication dispensing devices may be securely installed at a location and may be constructed of strong materials. In enclosed embodiments, the patient entry points may include locked doors. The medication storage units in which the medications are stored may be connected to a spoiling material, such that if the device detects an attempted theft, it may release the spoiling material to contact the stored medication such that the stored medication would be ruined and unusable.

Some medications, such as methadone or suboxone, need to be consumed immediately to prevent diversion. Therefore, various embodiments may include systems for ensuring that the patient consumes the medication before leaving. If the device detects that the patient did not consume the medication prior to leaving, it may automatically send a notice, such as to a health care provider such as a nurse, doctor, therapist, and/or pharmacists, and/or may refuse to dispense medication to the patient the next time the patient attempts to receive medication from the device.

The medication dispensing device may confirm that medication is immediately consumed by the patient, prior to departure or leaving the patient area, through observation of the patient. For example, the medication dispensing device may include a video camera for watching the patient and/or recording the patient encounter. In some embodiments, the live video stream and/or the video recording may connect to a remote observer with access to the video output of the camera who can visually ensure that the patient consumed the medication. This observation may be in real time or may be at a later time using a recording. In some embodiments, the medication which is dispensed may include smart technology for tracking of ingestion, which may communicate with a device worn by the patient such as a patch, which may in turn communicate with the patient's smart device and/or directly with the medication dispensing device. In some embodiments, the medication dispensing device may include software, or may be in communication with a remote software system such as a cloud-based system, which may automatically determine whether or not the patient consumed the medication.

The medication dispensing devices may include computer hardware and software components to control the operations of the medication dispensing device including user interactions with the device, dispensing of the medication, and tamper/theft detection, such as controllers, processors, servers, memory, internet communication elements, and other computer components. The medication dispensing devices may be configured for electronic communication outside the medication dispensing device through a hard-wired connection and/or a wireless connection to the internet, for example.

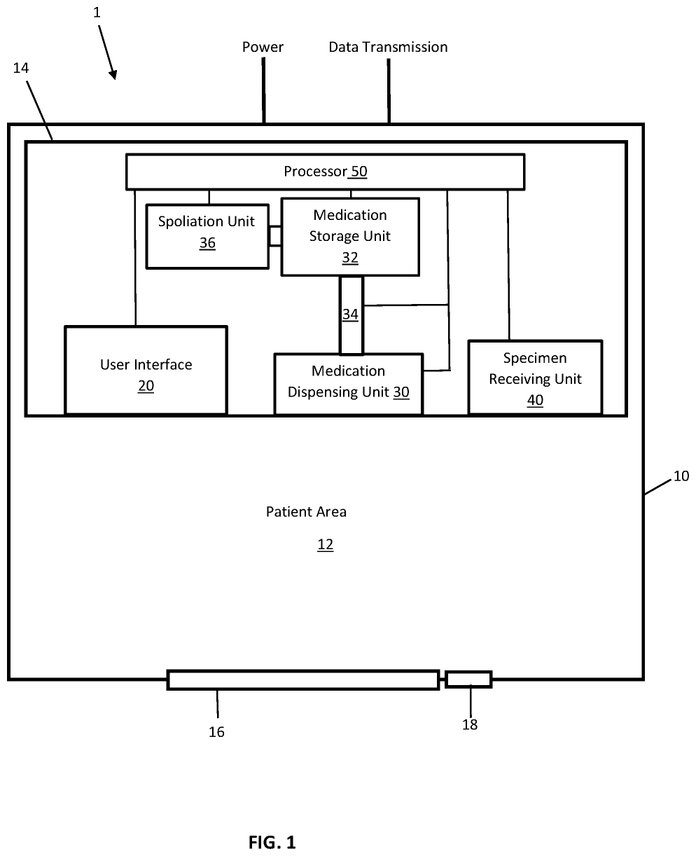

A schematic representation of a medication dispensing device is shown in FIG. 1. In this embodiment, the medication dispensing device 1 includes an enclosure 10 surrounding a patient area 12 and a machine portion 14. However, in alternative embodiments, the medication dispensing device 1 may not include an enclosure 10 and therefore may not include a defined patient area 12 but rather may include only the machine portion 14. However, patients may still access and interact with the machine portion 14 from an area adjacent to the machine portion, like the patient area 12 but not enclosed.

The enclosure 10 may include a plurality of connected walls, such as 4 walls, and a ceiling and floor that entirely enclose the patient area 12 and the machine portion 14. Alternatively, the enclosure may not fully enclose the machine portion 14 but rather the enclosure may connect with the machine portion 14 such that an outer aspect of the machine portion 14 may form a portion of the outside of the medication dispensing device 14. In such embodiments, the patient area may still be fully enclosed by the enclosure 10 and the machine portion 14. The enclosure 10 may further include a door 16. The door may be a solid door and may include a locking mechanism 18, such as a keypad or scanner such as a fingerprint or retina scanner, to limit access to the patient area 12 to authorized patients and other authorized users such as technicians. In other embodiments, the door 16 may be flexible, such as a curtain, or may be a door without a lock, for purposes of providing privacy only, or may be an open pass through.

In other embodiments, the enclosure 10 may only partially surround the patient area, such as to provide partial privacy. For example, the enclosure 10 may include walls that surround all of the sides of the patient area 12 but may not include a ceiling and/or a floor or may include walls that only partially surround the patient area 12, such as on two sides of the patient area 12.

The enclosure may further include access points for power, such as through a power cord, and electronic data communication, such as through a data transmission cable.

The patient area 12 may be a space in which the patient may stand while interacting with the machine portion 14 and may optionally include a chair or bench. The machine portion 14 is oriented for interaction with a patient in the patient area 12.

The machine portion 14 may include all of the functional elements of the medication dispensing device 1 such as those indicated in FIG. 1, such as in embodiments which include only a machine portion 14. Alternatively, some functional elements shown in the machine portion 14 in FIG. 1 or described herein as being included in the machine portion 14 may be located elsewhere such as in the enclosure walls, ceiling, and/or floor or external to the medication dispensing device 1.

The machine portion 14 may include a user interface 20 through which a patient and other users such as technicians may communicate with the medication dispensing device 1. The user interface 20 may include a keyboard, a display screen such as a touch screen, a microphone, a speaker, a camera such as a video camera, and/or other features. It may further include an identification scanner such as a fingerprint or retina scanner. A patient may use the user interface 20 to input required data needed for patient identification, such as a name, number, fingerprint, retinal scan, voice scan, or facial image. In addition, the medication dispensing device may use the user interface 20 to communicate with the patient, such as by conveying written and/or video instructions such as by displaying them on the display screen and/or emitting verbal instructions. It may further provide two-way personal communication between the patient and an individual at a remote location though an audio and/or visual video link using the display screen, speakers, video and microphone.

In some embodiments, one or more patient identification elements of the patient interface may be provided by a patient's own mobile device such as a patient's smart phone, such as through a smart phone app. For example, the app may employ the smart phone's camera, microphone or fingerprint scanners to identify the patient in the same way as those elements would work when included in the medication dispensing device 1, such as through a remote observer or through facial or voice recognition technology or fingerprint matching. The smart phone may also be used to provide the patient's location, to confirm that the patient is at or generally in the close vicinity of the medication dispensing device 1 at the time of identity verification. After verification of patient identity and/or location, the app may provide the user with a unique identifier, such as a multidigit alphanumeric code, a bar code, or a QR code. The patient may input the unique identifier into the medication dispensing unit through the patient portal, for example, by manually entering the alphanumeric code or using a scanner to scan the alphanumeric code, bar code, QR code or other code. After receiving the correct unique identifier, the medication dispensing device 1 may use the unique identifier to confirm the patient's identity and may proceed with the next steps, such as asking the patient questions, requesting specimens, and/or providing medication. In some embodiments, the unique identifier may only be valid for a brief period of time to prevent it being shared, such as 1 minute or 5 minutes or 10 minutes.

The machine portion 14 further includes a medication dispensing unit 30 connected to a medication storage unit 32. The medication storage unit 32 may contain a supply of a medication in the appropriate form, such as a powder or liquid or pill including all pill forms such as capsules, tablets, and other pill forms. In some embodiments, such as embodiments used with powdered medications, the machine portion 14 may further prepare the medication for consumption, such as by hydrating the power to form a liquid prior to dispensing the medication. In such embodiments, the machine portion 14 may further include other components necessary for preparing the medication to be dispensed, such as a liquid reservoir and mixing elements.

The medication storage unit 32 is connected to the medication dispensing unit 30 by a metered connection 34 which controls the amount of medication released by the medication storage unit 32. The metered connection 34 ensures that only the appropriate medication dose is released for the particular patient.

The medication dispensing unit 30 releases the medication from the medication dispensing device 1 to the patient. For liquid medications, it may pour the medication into a cup. The patient may remove the medication from the medication dispensing unit 30. In embodiments which provide a dose of medication to be consumed immediately, the medication dispensing device 1 may monitor the patient to ensure that the patient consumes the medication prior to leaving. For example, an observer may watch the patient consume the medication through a camera in the user interface and/or a software system which is trained to correctly identify when a patient swallows medication and may analyze the video to determine whether or not the patient consumed the medication. Such software may be included in the medication dispensing device 1 or may be remote.

In some embodiments, technology such as smart pill technology may be used for patient ingestion monitoring. In such embodiments, such as those in which the medication which is dispensed is a pill or tablet, a sensor may be supplied in the medication, such as embedded within the pill or tablet. When swallowed, the sensor may detect its change in location to the stomach and may transmit this change in location and/or the change in its location may be detectable. For example, the sensor may change its state and be activated by biological material's present in a patient's gastrointestinal tract such as stomach acid or enzymes. Such sensors may be comprised of nontoxic materials such as copper, magnesium and silicon. For example, in some embodiments, the patient may wear a consumption detection device such as a patch, wrist band, or other device which detects when a pill with a sensor is swallowed, and which transmits this information to another device such as the patient's smart phone, such as through blue tooth transmission. The smart phone may then transmit this information to the medication dispensing device 1 or to a remote service in communication with the medication dispensing device 1 and/or the patient's pharmacy and/or medical providers. Embodiments in which the patient wears a consumption detection device may be particularly useful in embodiments in which more than one days of medication supplies are provided to a patient, to monitor compliance after leaving the medication dispensing unit 1. In some such embodiments, the wearable consumption detection device may not be removed by the patient without evidence of such removal, such as with wrist bands which must be cut to remove them, to ensure that the wearable consumption detection device was not used by another person. Alternatively, when the medication is meant to be consumed immediately, prior to leaving the medication dispensing unit 1, the patient may not wear a consumption detection device but rather the medication dispensing device 1 may include a consumption detection device which may directly detect the signal or change in state of the sensor within the medication when it is swallowed. Such systems may be used not only to confirm that a patient consumed a medication, but also when the medication was consumed and in what doses.

The machine portion 14 may further include a spoliation unit 36 in connection with the medication storage unit 32. The spoliation unit 36 may include a mechanism or material which may ruin the medication in the event an attempted theft of the medication or other tampering with the medication dispensing device 1.

The medication dispensing device further includes one or more optional specimen collection units 40. Some specimen collection units 40 may receive breath samples for breath analysis by a patient breaching into the unit 40. Other specimen collection units 40 may receive urine sample, such as samples provided by patients in containers. The specimen collection units 40 may not only receive the specimens but may also analyze them, such as to detect the presence and/or level of substances of concern such as alcohol or recreational drugs, the presence of which may prohibit the patient from receiving the medication.

The functions of the medication dispensing device 1 may be controlled through a central processing unit such as one or more processors 40 with appropriate memory, software and other elements, which communicate with and control the elements and their functioning in medication dispensing device 1 as described herein. The medication dispensing device 1 may further include other elements necessary to perform the functions described herein as understood in the art.

In some embodiments, the medication dispensing device 1 may dispense more than one dose of a medication. The multiple doses of medication may be dispensed together in a single container, such as a single pill bottle or single liquid bottle. Alternatively, the multiple doses of medication may be dispensed in multiple containers or multiple compartments of a single container, with each container or each container compartment including a single dose. In some embodiments, the multiple containers may be dispensed in a lock box, or the container including multiple compartments may be a lock box, such as for medications such as methadone or suboxone for which there is a risk of abuse or theft or for which a lock box is legally required. In some such embodiments, the medication dispensing device may dispense the medication into the multiple containers or compartments prior to dispensing to the patient. Alternatively, a pharmacist may prepare the multiple containers or compartments in a lock box for a patient in advance and the prefilled lockbox may be stored in the medication dispensing device until dispensed by the medication dispensing unit 30 and removed by the patient.

The lock box may be a secure box made of a strong material such as metal or plastic. It may have any suitable dimensions but may be sized to include multiple doses while still being portable and may optionally include a handle. For example, the lock box may measure between about 2 inches and about 18 inches per side, or between about 3 inches and about 12 inches per side. It may be secured closed with a lock and key mechanism, a combination lock, or other secure locking mechanism to limit access only to the patient.

In some embodiments, the lock box may include one or more components for monitoring patient compliance. For example, the lock box may include components which detect when the lock box is opened. If the lock box includes multiple compartments, such as one compartment for each dose, the lock box may include components which detect opening of the individual compartments. This information, which may include how many times and when the opening occurred, may be stored within a data storage device in the lock box and accessed by the medication dispensing unit 1 when the lock box is returned and/or by a patient's pharmacists and/or medical provider. Alternatively, or additionally, the lock box may include data transmission components configured to send compliance information, such as how many times and when the lock box and/or lock box compartments are opened, to a patient's cell phone, to a remote monitoring system and/or to a patient's pharmacist and/or medical provider.

In the foregoing description, the inventions have been described with reference to specific embodiments. However, it may be understood that various modifications and changes may be made without departing from the scope of the inventions.

What is claimed is:

1. A medication dispensing device comprising:
a medication storage unit;
a medication dispensing unit;
a user interface;
a user identification element; and
a processor;
wherein the medication dispensing device is configured to identify a patient and dispense one or more doses of a medication.

2. The device of claim 1 wherein the medication dispensing device is configured to dispense a single dose of the medication and is further configured to confirm that the patient consumed the single dose of medication immediately.

3. The device of claim 1 further comprising a medication spoliation unit, wherein the medication spoliation unit is configured to ruin the stored medication when an attempted theft is detected.

4. The device of claim 1 wherein the user identification element comprises a camera, fingerprint scanner, a retina scanner, a bar code scanner, or a QR code scanner.

5. The device of claim 1 further comprising a camera.

6. The device of claim 5 wherein the device includes data output through a wired or wireless connection, wherein the device is configured images or video of a patient interacting with the device or consuming the medication to a remote observer.

7. The device of claim 1 further comprising a specimen collection unit.

8. The device of claim 7 wherein the specimen collection unit is a breath analyzer.

9. A medication dispensing device comprising:
a machine portion comprising:
a medication storage unit;
a medication dispensing unit;
a user interface;
a user identification element; and
a processor; and
an enclosure defining a patient area;
wherein the medication dispensing device is configured to identify a patient and dispense one or more doses of a medication.

10. The device of claim 9 wherein the enclosure encloses the patient area.

11. The device of claim 9 wherein the enclosure further comprises a door secured by a lock providing controlled entry to the patient area.

12. The device of claim 11 wherein the lock comprises a user identification element.

13. The device of claim 12 wherein the user identification element comprises a fingerprint scanner, a retinal scanner, a key pad, a bar code scanner or a QR code scanner.

14. The device of claim 9 further comprising a medication spoliation unit, wherein the medication spoliation unit is configured to ruin the stored medication when an attempted theft is detected.

15. A method of dispensing medication comprising:
storing medication in a mediation dispensing device, the medication dispensing device comprising:
a medication storage unit;
a medication dispensing unit;
a user interface;
a user identification element; and
a processor;
receiving data from a patient;
confirming the patient identity using the received data; and
after confirming the patient identity, automatically dispensing medication to the patient.

16. The method of claim 15 further comprising receiving a specimen from a patient.

17. The method of claim 16 wherein receiving a specimen comprises receiving a breath sample or a urine sample.

18. The method of claim 16 further comprising analyzing the sample to obtain a result, wherein the step of automatically dispensing the medication to the patient only occurs if the result of the sample analysis falls within a predefined parameter.

19. The method of claim 15 wherein the device further comprises a video camera, the method further comprising receiving a video of the patient consuming the dispensed medication using the video camera.

20. The method of claim 19 further comprising transmitting the video to a remote observer.
